(19) United States
(12) Patent Application Publication
(10) Pub. No.: US 2006/0224165 A1
(43) Pub. Date: Oct. 5, 2006

(54) Multi-clip device

(75) Inventors: Vihar C. Surti, Winston-Salem, NC (US); Leonard Erickson, Lewisville, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc.

(21) Appl. No.: 11/371,369

(22) Filed: Mar. 8, 2006

Related U.S. Application Data
(60) Provisional application No. 60/661,288, filed on Mar. 11, 2005.

(51) Int. Cl. A61B 17/10

(52) U.S. Cl. 606/142

(57) ABSTRACT

A multi-clip device for hemostasis includes an introducing tube (outer sheath) insertable into a body cavity. An operating wire is disposed within the introducing tube, and is advanceable and retractable within the introducing tube. A plurality of detachable clips are arranged in a serial fashion within the introducing tube. Each clip has an engagement portion at the proximal end thereof and a plurality of arms extending towards the distal end thereof. The distal end of the operating wire engages the engagement portion of the proximal most clip, and the arms of the proximal most clip engage the engagement portion of the adjacent distally located clip. An expandable collar is slidably disposed about the arms of the clip and is configured such that when the collar is moved distally over the arms it closes and holds them in a closed position. The expandable collar has an expanding portion that is engaged by the distal end of the introducing tube so that the introducing tube can be used to advance the collar distally relative to the clip.

### RELATED APPLICATIONS

This application claims the benefit of U.S. Provisional Application No. 60/661,288, filed Mar. 11, 2005.

### TECHNICAL FIELD

The present invention relates to a multi-clip device, and more specifically to a device for delivering a plurality of clips that can be used to cause hemostasis of blood vessels along the gastrointestinal tract, or that can be used as an endoscopic tool for holding tissue or the like.

### BACKGROUND OF THE INVENTION

Conventionally, a clip may be introduced into a body cavity through an endoscope to grasp living tissue of a body cavity for hemostasis, marking, and/or ligating. In addition, clips are now being used in a number of applications related to gastrointestinal bleeding such as peptic ulcers, Mallory-Weiss tears, Dieulafoy's lesions, angiomas, post-papillotomy bleeding, and small varices with active bleeding.

Gastrointestinal bleeding is a somewhat common and serious condition that is often fatal if left untreated. This problem has prompted the development of a number of endoscopic therapeutic approaches to achieve hemostasis such as the injection of sclerosing agents and contact thermo-coagulation techniques. Although such approaches are often effective, bleeding continues for many patients and corrective surgery therefore becomes necessary. Because surgery is an invasive technique that is associated with a high mortality rate and many other undesirable side effects, there exists a need for highly effective less invasive procedures.

Mechanical haemostatic devices have been used in various parts of the body, including gastrointestinal applications. Such devices are typically in the form of clamps, clips, staples, sutures, etc. that are able to apply sufficient constrictive forces to blood vessels so as to limit or interrupt blood flow. One of the problems associated with conventional haemostatic devices, however, is that they can only be delivered using rigid shafted instruments via incision or trocar cannula. Moreover, none of the conventional haemostatic devices are strong enough to cause permanent hemostasis.

One proposed solution is described in U.S. Pat. No. 5,766,189, which shows a device for delivering a detachable clip having a pair of open arms that can be closed about the blood vessel. One problem with this clip device and other similar types of devices having detachable clips is that the device is only capable of delivering a single clip. Thus, if the blood vessel is relatively large, or if more than one blood vessel requires constriction, then a second (or third) clip device must be introduced into the target area within the patient, thereby increasing the complexity and duration of the medical procedure. Accordingly, there is a need for a clip device that can deliver a plurality of detachable clips to the target area without having to introduce multiple separate delivery devices, or without having to remove, reload, and then reintroduce the clip delivery device with a second (or third) detachable clip.

The multi-clip device of the present invention solves this and other problems by providing a clip delivery device capable of delivery a plurality of detachable clips to the target area within the patient.

Another problem with the clip described in U.S. Pat. No. 5,766,189 and other similar types of clips having a pair of arms is that it may often be necessary to rotate the clip to properly grasp the area to be clipped. Rotation of the clip is often hindered or complicated by the travel of the operating wire through the bends of the tube(s) used to deliver the clip. Accordingly, there is a need for a clip that can be delivered to the target area and used without having to rotate the clip to a desired orientation.

The multi-clip device of the present invention solves this and other problems by providing a detachable clip having at least three arms.

Another problem often encountered with conventional haemostatic devices is the difficulty in securing the clip device to the delivery apparatus prior to reaching the target area within the patient, and then quickly and easily releasing the clip device from the delivery apparatus once the clip has been attached to the target site.

The multi-clip device of the present invention solves this and other problems by providing a detachable clip having a retainer system that is secured by a sliding collar.

Another problem often encountered with conventional haemostatic devices is the difficulty in properly positioning the device to grasp the area to be clipped when the surgical site is obscured by blood or other bodily fluids. For example, when attempting to clip a bleeding vessel, the area surrounding the vessel is often filled with blood, thereby preventing the surgeon from being able to locate and/or clip the vessel. It is therefore usually necessary to flush the surgical site with saline so as to wash away any blood or other bodily fluids that may be obstructing the surgeon's visibility of the site. This procedure is ordinarily accomplished by the use of a separate catheter that has been inserted into the patient and directed to the surgical site. In addition to the need for a separate catheter, the procedure often results in delays in the clipping of the vessel because of the additional time required for inserting and positioning the catheter. Accordingly, there is a need for a clip that can be properly positioned in the target area without having to utilize a separately inserted catheter to flush the target area of blood or other bodily fluids.

The multi-clip device of the present invention solves this and other problems by providing a clip delivery device having an integrated flushing feature.

### SUMMARY OF THE INVENTION

Accordingly, the present invention provides a delivery device that is capable of delivering a plurality of detachable clips that can reliably grasp tissue within the body cavity of a patient during a medical procedure without injury.

According to one aspect of the present invention, an exemplary multi-clip device is provided and comprises an introducing tube that is insertable into the body cavity. Disposed within the introducing tube (also referred to as the sheath) is an operating wire (also referred to as a drive cable). The operating wire is independently slidable within the introducing tube. In other words, the operating wire can be advanced and retracted independently of the movement of the introducing tube.

A plurality of detachable clips is disposed within the introducing tube. Each clip has an engagement portion at the proximal end thereof and a plurality of arms extending towards the distal end thereof. The arms are formed of a resilient material and are shaped such that the arms have a tendency to be in an open position towards the distal end of the clip. The engagement portion is configured such that it can be engaged by the distal end of the operating wire or by the closed arms of another clip. In one embodiment the clip comprises a pair of arms, and in another embodiment the clip comprises three of more arms.

An expandable collar is slidably disposed about the arms of the clip and is configured such that when the collar is moved distally over the arms it closes and holds them in a closed position. The collar has an expanding portion that is movable from a first compressed position to second expanded position. When in the first compress position, the collar is slidably disposed within the introducing tube, and when in the second expanded position, the collar may be engaged by the distal end of the introducing tube so that the introducing tube can be used to advance the collar distally relative to the clip.

The clips are arranged inside the introducing tube in a serial fashion. The distal end of the operating wire engages the engagement portion of the proximal most clip. The arms of the proximal most clip engage the engagement portion of the adjacent distally located clip. Additional clips, if included, are connected to each other in the same “head-to-tail” fashion. In other words, each clip is connected to the operating wire via each of the proximally located intervening clips.

To deploy a clip, the operating wire is advanced distally relative to the introducing tube to extend the arms of the distal most clip out of the distal end of the introducing tube. The distal most clip is advanced far enough to allow the expandable portion of the collar to expand to its second configuration. Once the arms of the clip are positioned about the target tissue, the operating wire is retracted in a proximal direction (or the introducing tube is advanced in a distal direction) to engage the collar with the distal end of the introducing tube. Further proximal movement of the operating wire relative to the introducing tube causes the collar to slide over the arms so as to close the arms onto the tissue. The operating wire is then advanced in a distal direction (or the introducing tube is retracted in a proximal direction) to extend the arms of the next proximally located clip out of the distal end of the introducing tube a distance sufficient to open the arms and release the deployed clip. The delivery device can then be used to deploy another clip by following the same procedure.

According to another aspect of the present invention, a method of delivering a plurality of detachable clips as described above is provided. The method includes disposing a plurality of detachable clips within an introducing tube and connected to each other in a serial arrangement, wherein the proximal most clip is connected to an operating wire movably disposed within the introducing tube. The distal end of the introducing tube is positioned near the targeted tissue. The operating wire is then advanced to expose the distal most clip from the introducing tube. Once the exposed clip is position about the targeted tissue, the operating wire is retracted so as to cause the introducing tube to engage an expandable collar slidably attached to the clip and advance the collar over the arms of the clip. Advancement of the collar over the arms of the clip causes the arms to close onto or about the targeted tissue. Thereafter, the operating wire is advanced so as to expose and open the arms of the next proximally located clip, thereby releasing the deployed clip from the delivery device. The above steps are then repeated to deploy one or more additional detachable clips. The delivery device is then retrieved and the deployed clips are left behind.

An additional aspect of the present invention is to provide a multi-clip device having an integrated flushing feature. The flushing feature includes a port located in the forward portion of the handle. In one embodiment, the port is in fluid communication with the cavity or open volume within the introducing tube. In another embodiment, the port is in fluid communication with a separate lumen extending through the wall of the introducing tube. This cavity or lumen extends forward from the handle to near the distal end of the introducing tube. As a result, the injection of any fluid, such as saline solution, through the port in the handle is directed through the cavity or lumen and out the distal end of the introducing tube. The flushing feature permits the surgical site to be flushed of blood or other bodily fluids prior to and/or while positioning the clip to grasp the targeted tissue.

According to one method of using the flushing feature described above, the distal end of the clip delivery device is first delivered to the surgical site where the targeted tissue is generally located. If it is determined that the targeted tissue is obscured by blood or other bodily fluids, then saline is injected through the port in the handle so as to pass through the cavity or lumen of the introducing tube. The saline exits the cavity or lumen near the distal end of the introducing tube, thereby flushing the area surrounding the target site of any blood or other bodily fluids. Injection of saline through the port is continued or repeated as necessary to flush the surgical site during the treatment.

### BRIEF DESCRIPTION OF THE DRAWINGS

FIG. 1 is an illustration of one embodiment of the multi-clip device according to the present invention.

FIG. 2 is a cross-sectional illustration of the distal portion of the multi-clip device of FIG. 1 before deployment of a detachable clip.

FIG. 3 is a cross-sectional illustration of the distal portion of the multi-clip device of FIG. 1 during a first stage of deployment of a detachable clip.

FIG. 4 is a cross-sectional illustration of the distal portion of the multi-clip device of FIG. 1 during a second stage of deployment of a detachable clip.

FIG. 5 is a cross-sectional illustration of the distal portion of the multi-clip device of FIG. 1 after deployment and release of a detachable clip.

FIG. 6 is a perspective view of a first embodiment of a detachable clip of the present invention.

FIG. 7 is a perspective view of a second embodiment of a detachable clip of the present invention.

FIG. 8 is a perspective view of an embodiment of an expandable collar of the present invention.

### DESCRIPTION OF THE INVENTION

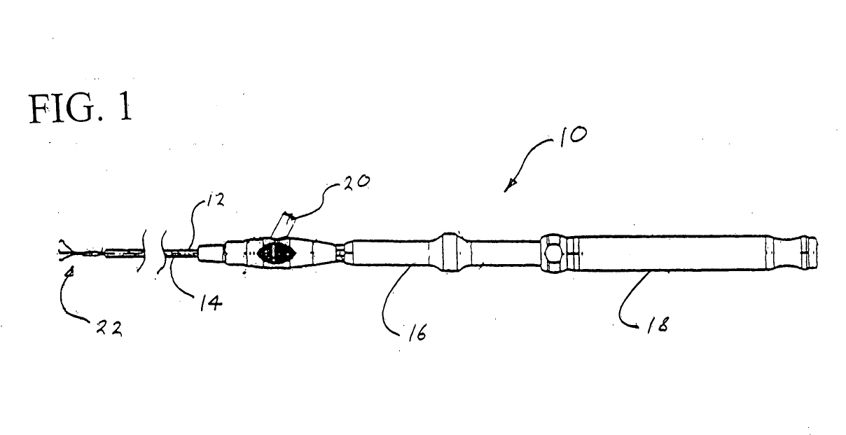

The present invention provides a multi-clip device for grasping tissue or the like. Referring to FIG. 1, an exemplary multi-clip device according to the present invention is shown. The multi-clip delivery device 10 includes an introducing tube 12 that is insertable into the body cavity of a patient. Disposed within the introducing tube 12 (also referred to as the sheath) is an operating wire 14 (also referred to as a drive cable). The operating wire 14 is independently slidable within the introducing tube 12. In other words, the operating wire 14 can be advanced and retracted independently of the movement of the introducing tube 12.

The introducing tube 12 is attached at its proximal end to a forward handle portion 16. The operating wire 14 extends through the forward handle portion 16 and is attached at its proximal end to a rearward handle portion 18, which is disposed proximally of the forward handle portion 16. The rearward handle portion 18 telescopically extends over the proximal portion of the forward handle portion 16. As will be explained in more detail below, longitudinal movement of the operating wire 14 relative to the introducing tube 12 is controlled by longitudinal manipulation of the forward handle portion 16 relative to the rearward handles portion 18.

The forward handle portion 16 also includes a flushing port 20. The flushing port 20 can comprise a standard male or female luer fitting, or any other valve mechanism that permits the injection of fluid there through. The flushing port 20 is in fluid communication with the interior volume of the forward handle portion 16, which in turn is in fluid communication with the cavity or lumen within the introducing tube 12. Accordingly, any fluid injected through the flushing port 20 will necessarily enter the cavity or lumen of the introducing tube 12, and will subsequently exit the cavity near the distal end of the introducing tube 12. In other words, the fluid injected through the flushing port 20 will exit the distal end of the multi-clip device 10.

Alternatively, the cavity or lumen can be disposed within the wall of the introducing tube 12. In other words, the introducing sheath can comprise a separate lumen disposed there through which fluid can be passed along the length thereof. In another alternative embodiment, the lumen can be disposed through the operating wire 14. It should also be understood that the flushing port 20 could be alternatively located on the rearward handle portion 18, or on a portion of the introducing tube 12 distally of any of the handle portions.

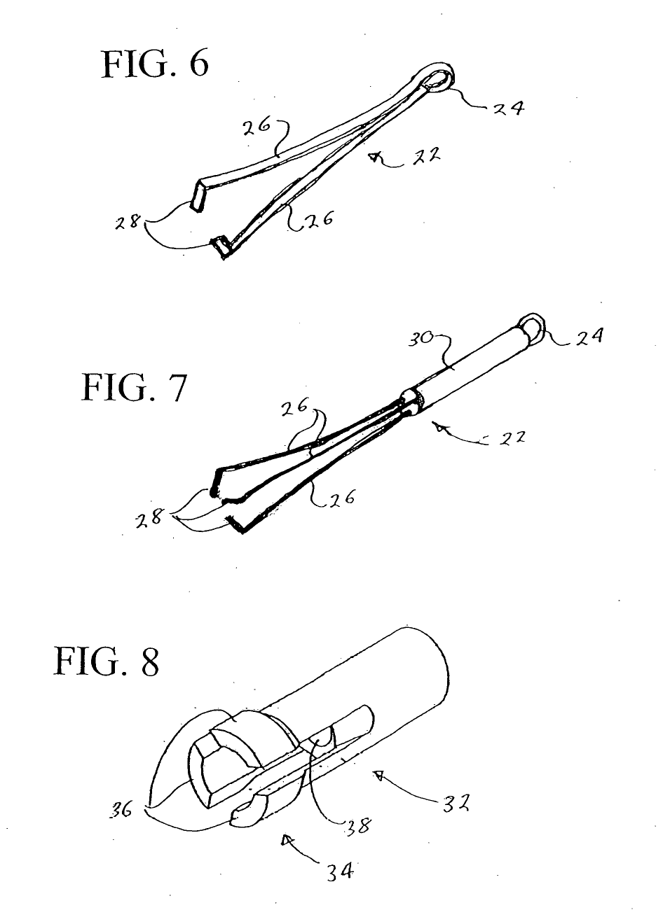

The multi-clip delivery device 10 further includes a plurality of detachable clips 22 disposed within the introducing tube 12. As illustrated in FIGS. 6 and 7, each clip 22 has an engagement portion 24 at the proximal end thereof and a plurality of arms 26 extending towards the distal end thereof. The arms 26 are formed of a resilient material and are shaped such that the arms 26 have a tendency to be in an open position towards the distal end of the clip 22. Each arm 26 includes an inwardly facing prong or tooth 28 which is configured to grasp the target tissue. The teeth 28 preferably overlap with each other when the clip 22 is in the closed position. The length of the individual arms 26 may be different to provide for overlapping of the teeth 28. As will be explained in greater detail below, the engagement portion 24 is configured such that it can be engaged by the distal end of the operating wire 14 or by the closed arms 26 of another clip 22.

In the embodiment illustrated in FIG. 6, the clip 22 comprises a pair of arms 26 formed by bending a single elongate piece of resilient material. The engagement portion 24 is likewise formed by bending the central portion of the elongate piece of resilient material to form a loop.

In the embodiment illustrated in FIG. 7, the clip 22 comprises three arms 26. Each arm 26 is individually formed from a resilient material and affixed to a central tube 30 by any suitable means such as welding or gluing. The engagement portion 24 is likewise individually formed and attached to a proximal end of the central tube 30. The use of three arms allows the clip 22 to grasp the target tissue with minimal, if any, need to rotate the clip 22 into the correct orientation. While three arms are illustrated in this embodiment, it is contemplated that more than three arms may be used.

The clip 22 may be made from any suitable resilient material such as stainless steel, nitinol, plastic, and the like, and is preferably a biocompatible material. The material used for the clip 22 may also be bio-degradable. The use of a bio-degradable material will allow the clip 22 to detach itself from the target tissue after a desired period of time and pass naturally out of the patient's body. In addition, the arms may have a cross-sectional shape that is round, square, triangular, pie-shaped, truncated cone, and the like. For example, the arms 26 of the clip 22 illustrated in FIG. 6 have a rectangular, flat bar-like cross-section, whereas the arms 26 of the clip 22 illustrated in FIG. 7 have a round, wire-like cross-section. A triangular or delta shaped cross-section is particularly advantageous for a clip having three arms because it allows a reduction in the cross-sectional area that the arms occupy within the introducing tube 12, thereby allowing a reduction in the diameter of the introducing tube 12.

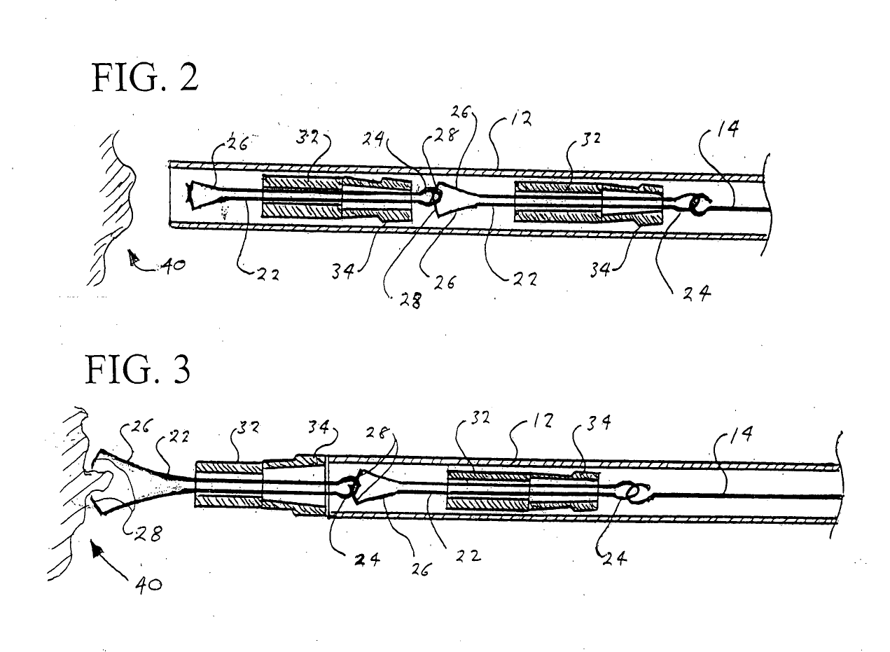

An expandable collar 32 is slidably disposed about the arms 26 of the clip 22 and is configured such that when the collar 32 is moved distally over the arms 26 it closes and holds the arms 26 in a closed position. As illustrated in FIG. 8, the collar 32 has an expanding portion 34 that is movable from a first compressed position to second expanded position. When in the first compress position, the collar 32 may be slidably disposed within the introducing tube 12 (FIG. 2), and when in the second expanded position (FIG. 3), the collar 32 may be engaged by the distal end of the introducing tube 12 so that the introducing tube 12 can be used to advance the collar 32 distally relative to the clip 22.

In the embodiment illustrated in FIG. 8, the expanding portion 34 comprises a plurality of movable arms 36 extending towards the proximal end of the collar 32. The movable arms 36 are biased in an outward transverse direction so as to have an outer diameter that is greater than the inside diameter of the introducing tube 12. However, and as will be explained in greater detail below, the movable arms 36 are compressible to allow the collar 32 to slidably fit within the introducing tube 12. In the alternative, the expanding portion 34 may comprise a resilient material, such as rubber, that is compressible from the expanded position to the compressed position. Such an embodiment would not require any movable components.

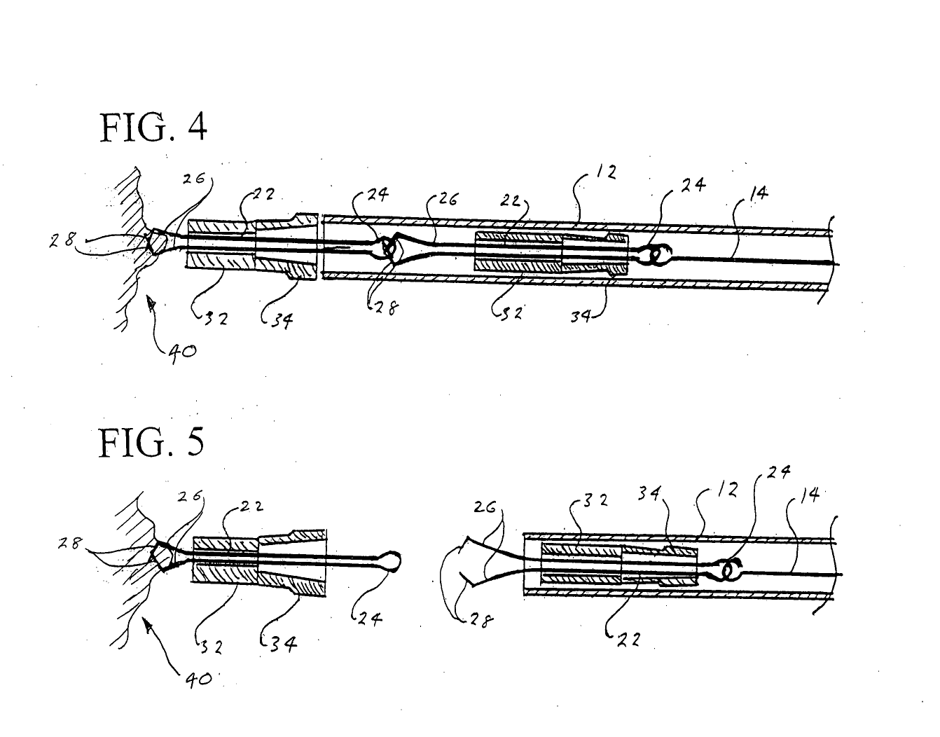

The collar 32 further comprises a central lumen 38 through which the clip 22 is slidably disposed. The central lumen 38 is sized so as to close the arms 26 of the clip 22 as the collar 32 is advanced towards the distal end of the clip 22 (see FIGS. 4 and 5). In other words, the wall portion of the collar 32 that defines the central lumen 38 is configured to engage and overcome the transverse outwardly directed biasing force of the arms 26 so as to push the teeth 28 of the arms 26 into a closed and/or overlapping arrangement as the collar 32 is moved distally relative to the clip 22. The central lumen 38 may also be sized so as to prevent the proximal end of the clip 22, i.e., the engagement portion 24, from passing there through so as to prevent the collar 32 from separating from the clip 22.

The exterior surface of the collar 32 may also be sized and/or configured to allow the passage of fluids around or through the collar 32. As will be explained below, it may be desirable, for example, to pass saline through the introducing tube 12 to flush any blood or bodily fluids away from the part to be treated. Thus, the collar 26 may be sized to provide a gap between the exterior surface of the collar 26 and the interior surface of the introducing tube 12 through which fluids can pass. Alternatively, the collar 26 may include a flow channel or lumen extending there through.

The expandable collar 32 may be made from any suitable resilient material such as plastic, rubber, stainless steel, nitinol, and the like, and is preferably a biocompatible material. The material used for the collar 32 may also be biodegradable. The expandable collar 32 can be manufactured by any suitable procedure, such as milling (in the case of metal materials) or injection molding (in the case of plastic and rubber materials). While three movable arms 36 are illustrated in the embodiment shown in FIG. 8, it is contemplated any number of movable arms or components may be used. For example, the collar 32 may comprise a single arm or other type of movable or expandable device that can extend outwardly in a transverse direction so as to be engaged by the distal end of the introducing tube 12.

As best seen in FIG. 2, a plurality of clips 22 are arranged inside the introducing tube 12 in a serial fashion. The distal end of the operating wire 14 engages the engagement portion 24 of the proximal most clip 22 (the right-most clip 22 in FIG. 2). The arms 26, and more specifically the teeth 28, of the proximal most clip 22 engage the engagement portion 24 of the adjacent distally located clip 22 (the left-most clip 22 in FIG. 2). The inside surface of the introducing tube 12 maintains the arms 26 in a closed position through or about the engagement portion 24 of the adjacent clip. The expandable collar 32 helps to maintain the position of each clip 22 within the introducing tube 12.

Although the embodiment illustrated in FIG. 2 includes two clips 22, any number of clips 22 can be loaded into the introducing tube 12 of the multi-clip device 10. Additional clips (not shown) may be connected to each other in the same “head-to-tail” fashion as shown in FIG. 2. In other words, each clip 22 is connected to the operating 14 wire via each of the proximally located intervening clips. Thus, each intervening clip 22 functions as an extension of the operating wire 14. This arrangement eliminates the need for separate operating wires connected to each of the individual clips.

The operation of the multi-clip device 10 will be described. Prior to introduction of the device 10 into the patient, a plurality of clips 22 are disposed in the introducing tube 12 and connected to the operating wire 14 as shown in FIG. 2. This may be accomplished by connecting the engagement portion 24 of a first clip 22 to the operating wire 14, and then retracting the operating wire 14 so as to draw the first clip 22 into the introducing tube 12. The expandable collar 32 of the first clip 22 must be compressed so as to allow the collar 32 to be drawn into the introducing tube 12. The engagement portion 24 of a second clip 22 is then connected to the arms 26 of the first clip 22. The operating wire 14 is then further retracted so as to draw the second clip 22, along with its expandable collar 32, into the introducing tube 12. This procedure is repeated until the desired number of clips 22 are loaded into the introducing tube 12 of the device 10.

The introducing tube 12 is then introduced into a body cavity via the channel of an endoscope or similar device that has been previously inserted into the body cavity. While the body cavity is observed via the endoscope, the distal end portion of the outer introducing tube 12 is guided to the part to be treated.

If the part to be treated is obscured by blood or other bodily fluids, then a fluid such as saline is injected through the flushing port 22 on the forward handle portion 16. The saline enters the cavity or lumen within the introducing tube 12 and exits the distal end thereof. The saline floods the area so as to flush any blood or bodily fluids away from the part to be treated. The injection of saline is continued and/or repeated as necessary during the remaining steps of the procedure (described below) so as to keep the area free of blood and other bodily fluids.

Alternatively, a vacuum is applied to the flushing port 22 so as to create suction within the cavity or lumen within introducing tube 12. This suction can be used to remove blood or other bodily fluids from the area surrounding the part to be treated.

To deploy the clip 22, the operating wire 14 is advanced distally relative to the introducing tube 12 to extend the arms 26 of the distal most clip 22 out of the distal end of the introducing tube 12, thereby allowing the arms 26 to assume their expanded, open configuration. As shown in FIG. 3, the distal most clip 22 (the left-most clip 22) is also advanced far enough to allow the expandable portion 34 of the collar 32 to expand to its second expanded configuration. The extended clip 22 can now be positioned about the target tissue 40.

Once the arms 26 of the clip 22 are positioned about the target tissue 40, the operating wire 14 is retracted in a proximal direction (or the introducing tube 12 is advanced in a distal direction) so as to engage the collar 32 with the distal end of the introducing tube 12. More specifically, the proximal movement of the operating wire 14 relative to the introducing tube 12 causes the distal end of the introducing tube 12 to engage the expanding portion 34 of the collar 32. As shown in FIG. 4, further proximal movement of the operating wire 14 relative to the introducing tube 12 causes the collar 32 to slide over the arms 26 of the clip 22 so as to close the arms 26 onto the tissue 40. In other words, as the clip 22 is being pulled or drawn proximally into the introducing tube 12, the expanding portion 34 prevents the collar 32 from being likewise pulled or drawn into the introducing tube 12 as the clip 22. The result is that the collar 32 is pushed distally relative to the clip 22.

Once the clip 22 is secured to the target tissue 40 (i.e., deployed), the operating wire 14 is advanced in a distal direction (or the introducing tube 12 is retracted in a proximal direction) to release or detach the deployed clip 22 from the delivery device 10. More specifically, and as shown in FIG. 5, the operating wire 14 is advanced in a distal direction relative to the introducing tube 12 so as to push the next proximally located clip 22 (the right-most clip 22) and collar 32 assembly towards the distal end of the introducing tube 12. Once the arms 26 of the next proximally located clip 22 have been extended out of the distal end of the introducing tube 12 a distance sufficient to open the arms 26, the arms 26 open to disengage from the engagement portion 24 of the deployed clip 22, thereby releasing the deployed clip 22 from the delivery device 10. The delivery device 10 can then be used to deploy another clip 22 by following the same procedure described above.

It should be understood that any number of clips 22 can be initially loaded into the introducing tube 12. Each of these clips 22 would then be available for deployment during the medical procedure without the need to withdraw the multi-clip device 10 for re-loading after each deployment, and without the need to insert additional clip delivery devices.

In another embodiment of the multi-clip device 10, the proximal most clip 22 is permanently affixed to the operating wire 14. In other words, the proximal most clip 22 is not detachable. This non-detachable clip 22 would then be available to function as a forceps for retrieving objects, such as a previously deployed clip 22, or to take tissue samples from the target site. The non-detachable clip could also be used to manipulate and re-position a previously deployed clip 22.

While there have been described what are presently believed to be the preferred embodiments of the invention, those skilled in the art will realize that changes and modifications may be made thereto without departing from the spirit of the invention. It is to be understood that the invention can be carried out by specifically different equipment and devices, and that various modifications, both as to the equipment details and operating procedures, can be accomplished without departing from the scope of the invention itself.

1. A multi-clip device for use in endoscopic medical procedures comprising:
a) an introducing tube;
b) an operating wire disposed within the introducing tube, the operating wire being advanceable and retractable relative to the introducing tube; and
c) a plurality of detachable clips, each clip comprising an engagement portion, a plurality of arms, and a slidable collar disposed about the plurality of arms, said slidable collar being movable between a first position when said clip is in an open position and a second position when said clip is in a closed position, said slidable collar being configured to cause a distal end of the plurality of arms to be move towards each other when in the second position, the slidable collar comprising an expandable portion being movable between a compressed position when said clip is disposed within the introducing tube and an expanded position when said clip is at least partially disposed beyond a distal end of the introducing tube, said expandable portion being configured to be engaged by the distal end of the introducing tube when in the expanded position,
wherein the plurality of detachable clips comprises at least a distal clip and a proximal clip disposed in the introducing tube and arranged in a serial fashion such that the distal clip is disposed distally of the proximal clip,
further wherein the engagement portion of the distal clip is releasably engaged by one or more of the arms of the proximal clip, and the engagement portion of the proximal clip is operably connected to the operating wire.

2. The multi-clip device according to claim 1, wherein the expandable portion comprises a plurality of movable arms biased in a transverse outward direction, said movable arms having diameter that is equal or less than an interior diameter of the introducing tube when in the compressed position, and having a diameter that is greater than the interior diameter of the introducing tube when in the expanded position.

3. The multi-clip device according to claim 1, wherein the detachable clips each comprise at least three arms.

4. The multi-clip device according to claim 1, wherein each of the plurality of the arms of each of the detachable clips comprises an inwardly directed tooth for engaging a target tissue.

5. The multi-clip device according to claim 1, wherein each of the plurality of the arms of each of the detachable clips is biased in a transverse outward direction.

6. The multi-clip device according to claim 1, wherein the engagement portion of the distal clip comprises a loop configured to be releasably engaged by an inwardly directed tooth disposed on a distal end at least one of the plurality of arms of the proximal clip.

7. The multi-clip device according to claim 1, wherein the engagement portion of the proximal clip is affixed to the operating wire.

8. The multi-clip device according to claim 7, wherein the proximal clip comprises a forceps.

9. The multi-clip device according to claim 1, wherein the plurality of detachable clips comprises a third clip disposed in the introducing tube and arranged in a serial fashion such that the third clip is disposed distally of the distal clip, further wherein the engagement portion of the third clip is releasably engaged by one or more of the arms of the distal clip.

10. The multi-clip device according to claim 1, wherein the plurality of detachable clips comprises a third clip disposed in the introducing tube and arranged in a serial fashion such that the third clip is disposed proximally of the proximal clip, further wherein the engagement portion of the proximal clip is connected to the operating wire via the third clip.

11. The multi-clip device according to claim 1, wherein the expandable portion of the slidable collar comprises one or more flexible arms that are movable between the compressed position when said clip is disposed within the introducing tube and the expanded position when said clip is at least partially disposed beyond a distal end of the introducing tube.

12. The multi-clip device according to claim 1, wherein the slidable collar comprises a lumen through which the arms of the clip are slidably disposed.

13. The multi-clip device according to claim 12, wherein the lumen is defined by an interior surface of the collar that is configured to engage an outer surface of the arms of the clip when the clip is in the closed position.

14. The multi-clip device according to claim 12, wherein the lumen of the collar comprises a cross-sectional area that is smaller than a cross-sectional area of the engagement portion of the clip so as to prevent the collar from being removed from the proximal end of the clip.

15. The multi-clip device according to claim 1 further comprising a handle having a first handle portion and a second handle portion movable relative to the first portion, the first handle portion being connected to the introducing tube and the second handle portion being connected to the operating wire, wherein manipulation of the first handle portion relative to the second handle portion causes the operating wire to advance or retract relative to the introducing tube.

16. The multi-clip device according to claim 15, wherein the handle further comprises a port configured for the passage of fluids therethrough.

17. The multi-clip device according to claim 16, wherein the port is in fluid communication with the distal end of the introducing tube.

18. The multi-clip device according to claim 17, wherein port is in fluid communication with a lumen of the introducing tube.

19. The multi-clip device according to claim 17, wherein the slidable collar comprises a pathway for the passage of fluids between the port and the distal end of the introducing tube.
